(19) United States
(12) Patent Application Publication
(10) Pub. No.: US 2006/0116700 A1
(43) Pub. Date: Jun. 1, 2006

(54) Aortic stenosis cutting balloon blade

(76) Inventor: Loren M. Crow, La Mesa, CA (US)

Correspondence Address: NEIL K. NYDEGGER;NYDEGGER & ASSOCIATES, 348 Olive Street, San Diego, CA, 92103, US

(21) Appl. No.: 10/998,454

(22) Filed: Nov. 29, 2004

(51) Int. Cl. A61B 17/22

(52) U.S. Cl. 606/159; 606/194; 606/167

(57) ABSTRACT

A cutting device having a blade for incising internal tissue (e.g. a stenosis) of a patient includes an elongated balloon catheter, with at least one straight blade mounted on the balloon. The blade includes a blunt tip member at the blade's distal end to prevent tissue incision by the blade's distal tip. A proximal portion of the blade is attached to a proximal balloon section, and in operation, the balloon/blade combination is advanced into the vasculature and positioned distal to the stenosis to be incised. The balloon is then inflated. With this inflation, the blade is inclined relative to the axis of the catheter with an increasing distance between the blade and the axis in a distal direction. The device is then withdrawn, proximally, to incise the stenosis. The blunt distal blade tip reduces inadvertent incision of non-target tissue.

### FIELD OF THE INVENTION

The present invention pertains generally to interventional medical devices. More particularly, the present invention pertains to catheters that can be used to incise target tissue in the vasculature of a patient while minimizing collateral damage to non-target tissue. The present invention is particularly, but not exclusively, useful for incising an aortic valve stenosis with a catheter having a blade to prevent the inadvertent incision of non-target tissue.

### BACKGROUND OF THE INVENTION

The aortic valve controls the flow of oxygen-rich blood from the left ventricle into the aorta. Anatomically, the aortic valve consists of three semilunar cusps (i.e. right, left and posterior cusps) that are attached around the circumference of an opening that is located between the aorta and left ventricle. During each heart cycle, the cusps (also called flaps or leaflets) fold back against the inside wall of the aorta as the left ventricle contracts, effectively opening the aortic valve to allow blood to be pumped through the aorta and into the arteries in the vasculature of the body. Between contractions of the left ventricle, however, the cusps extend into the passageway between the left ventricle and aorta to close the aortic valve and form a tight seal that prevents blood from leaking back into the left ventricle from the aorta.

For any of several reasons (e.g. aging, or birth defects), it can happen that the aortic valve is somehow damaged and may become stenosed. When this happens, the aortic valve does not open to its normal extent and the flow of blood from the heart into the aorta is constricted. This leads to an undesirable heart condition that is commonly known as aortic valve stenosis (AS). If left untreated, AS can worsen and lead to a number of complications including endocarditis, arrhythmia and in some cases heart failure.

Heretofore, the conventional methods used to treat AS have typically involved either an aortic valve replacement or a procedure commonly known as percutaneous balloon valvuloplasty. In the case of a valve replacement, an extensive surgical procedure is generally required wherein the aortic valve is replaced either by a mechanical or a porcine valve. On the other hand, being a percutaneous procedure, balloon valvuloplasty is somewhat less involved than a valve replacement procedure. Nevertheless, for many reasons including a high recurrence rate, and despite its initial acceptance, balloon valvuloplasty is now used infrequently and only palliatively or as a bridge to a subsequent valve replacement.

More recently, efficacious treatments for aortic valve stenosis have been developed which entail incising and dilating the stenosed aortic valve. For example, a device and method for treating AS is disclosed in co-pending, co-owned U.S. patent application Ser. No. 10/353,827, filed by Leonard Schwartz (Schwartz '827) on Jan. 27, 2003, for an invention titled “A Device for Percutaneous Cutting and Dilating a Stenosis of the Aortic Valve”, and which is hereby incorporated by reference herein.

The present invention is directed to efficacious percutaneous devices and methods for treating a stenosis in a body conduit and is particularly applicable to the treatment of a valvular stenosis.

### SUMMARY OF THE INVENTION

In accordance with the present invention, a cutting device having a blade for treating a stenosis in a lumen or a valvular stenosis includes a catheter having an elongated balloon mounted near its distal end. As intended for the present invention, the balloon can be reconfigured on the catheter between an inflated configuration and a deflated configuration. Structurally, the balloon defines an axis and, in its inflated configuration, it has at least three identifiable sections that are located between its proximal end and its distal end. These sections are: a substantially conical-shaped proximal section having a taper with an increasing radius in the distal direction; a substantially conical-shaped distal section having a taper with a decreasing radius in the distal direction; and a substantially cylindrical-shaped intermediate section that is located between the proximal section and the distal section.

One or more elongated blades, that are typically substantially straight, are attached to the balloon. For the present invention, each blade is formed with a sharp edge and extends from a distal blade end to a proximal blade end. In a particular embodiment of the cutting device, a proximal portion of each blade is attached to the proximal section of the balloon. On the other hand, for this embodiment, the distal end of each blade is detached from the balloon to allow the blade to incline relative to the balloon axis when the balloon is inflated.

To reduce the likelihood of inadvertent tissue incision by the distal end of each blade during a movement of the blade, each blade has a blunt tip member at the blade's distal end. The blunt tip member can be attached to the portion of the blade having the cutting edge or integrally formed thereon. For example, in one embodiment of the present invention, the blunt tip member includes a fine coil wire and ball shaped element. More particularly, a fine coil wire having a distal end and a proximal end is provided, with the ball shaped element located at the distal end of the coil wire. In another embodiment of the present invention, the blunt tip member includes a protective sheath. For this embodiment, the cutting edge extends from a cutting edge distal end to a cutting edge proximal end and the protective sheath is positioned to overlay the distal end of the cutting edge. For example, the protective sheath can be made of plastic and bonded to the portion of the blade having the cutting edge. In yet another embodiment, the blunt tip member is formed as a rounded surface to prevent tissue incision by the distal tip of the blade.

In the operation of the present invention, the balloon (in its deflated configuration) is advanced into the vasculature of the patient. Specifically, for one exemplary treatment wherein AS is treated, the balloon is routed through the aorta and positioned inside the left ventricle of the heart. This then places the balloon distal to the aortic valve. Once the balloon is in the left ventricle it is then inflated.

In its inflated configuration, the balloon inclines each blade relative to the axis of the balloon. Specifically, this inclination is characterized by an increasing distance between the blade and the axis of the balloon, in a distal direction along the axis. In cooperation with the balloon, each blade is inclined relative to the balloon's axis at an angle (α) that is established by the taper of the balloon's proximal section, when the balloon is inflated. Preferably, this angle (α) is in a range between approximately zero degrees, when the balloon is in its deflated configuration, and approximately forty-five degrees, when the balloon is in its inflated configuration, (0°-45°). As a consequence of this cooperation of structure, when the balloon is in its inflated configuration, the sharp edges of the blade(s) are presented for cutting (incising) the aortic valve. More specifically, the distal portions and distal ends (including the blunt tip member) of the respective blade(s) are projected radially outward from the axis through a distance that extends beyond the radius of the cylindrical-shaped intermediate section.

An incising action on the aortic valve is accomplished as the inflated balloon is withdrawn through the aortic valve in a proximal direction. After the inflated balloon has been withdrawn through the aortic valve, and the valve has been incised, the balloon is deflated, retracting each blade into its original, non-inclined orientation. The deflated balloon and retracted blade(s) are then removed from the vasculature to complete the procedure.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of the incising device of the present invention;

FIG. 2A is a side view of the balloon of the device of the present invention when the balloon is in its deflated configuration;

FIG. 2B is a side view of the balloon of the device of the present invention when the balloon is in its inflated configuration;

FIG. 3A is an end view of the balloon of the device of the present invention as seen along the line 3A-3A in FIG. 2A;

FIG. 3B is an end view of the balloon of the device of the present invention as seen along the line 3B-3B in FIG. 2B;

FIG. 4 is a cross sectional view of the catheter as seen along the line 4-4 in FIG. 1;

FIG. 5 is an enlarged perspective view of the blade shown in FIG. 1;

FIG. 6 is an enlarged perspective view of another embodiment of a blade for use in the present invention having a fine coil wire and ball shaped element at the distal end;

FIG. 7 is an enlarged perspective view of another embodiment of a blade for use in the present invention having a substantially spherical blunt tip member attached distal to the blade's cutting edge;

FIG. 8 is an enlarged perspective view of another embodiment of a blade for use in the present invention having a blunt tip member formed as a rounded surface distal to the blade's cutting edge; and

FIG. 9 is a schematic view of an inflated balloon of the present invention, positioned inside the left ventricle of a patient, ready to be withdrawn in a proximal direction through an aortic valve for the purpose of incising the aortic valve.

### DESCRIPTION OF THE PREFERRED EMBODIMENTS

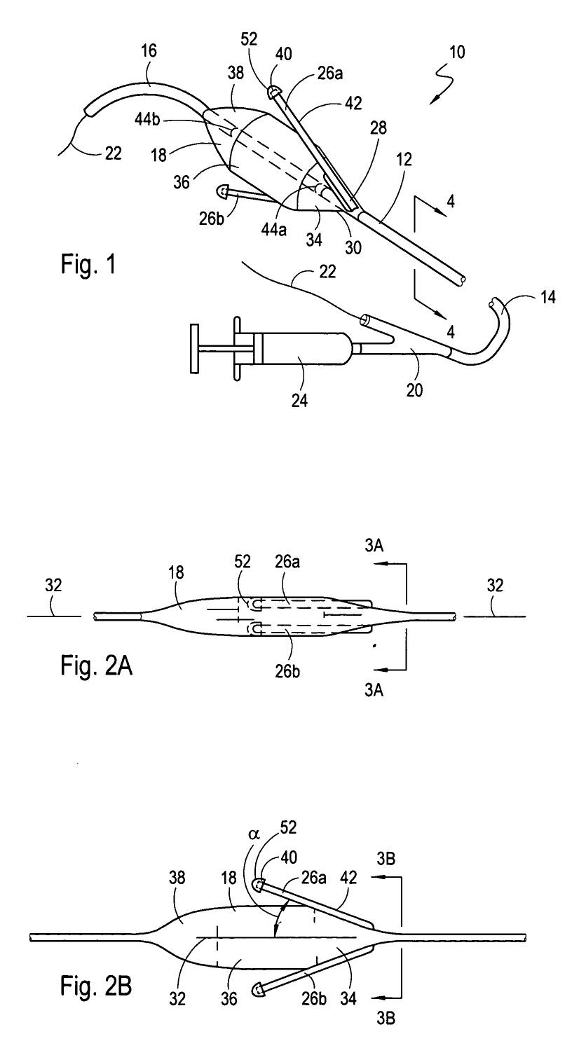

Referring initially to FIG. 1, a system for incising tissue in accordance with the present invention is shown and generally designated 10. As shown, the system 10 includes a catheter 12 which has a proximal end 14 and a distal end 16. System 10 also has an inflatable, elongated balloon 18 that is mounted on the catheter 12 near its distal end 16. Further, it is seen that a y-site 20 is attached to the proximal end 14 of the catheter 12. Specifically, the y-site 20 allows the catheter 12 to be operationally engaged with a guidewire 22 for the purpose of advancing the catheter 12 over the guidewire 22 after the guidewire 22 has been pre-positioned in the vasculature of a patient (not shown). FIG. 1 also shows that an inflation/deflation device 24 can be connected to the y-site 20 for fluid communication with the balloon 18.

For the catheter 12, the inflatable balloon 18 can be made of a compliant, semi-compliant or non-compliant material. Specifically, any suitable thermoplastic or thermosetting material may be used in accordance herewith including both elastomeric and non-elastomeric materials. Thermoplastic materials find particular utility herein. Examples of non-elastomeric materials include, but are not limited to, polyolefins including polyethylene and polypropylene, polyesters, polyethers, polyamides, polyurethanes, polyimides, and so forth, as well as copolymers and terpolymers thereof. As used herein, the term “copolymer” shall hereinafter be used to refer to any polymer formed from two or more monomers.

Examples of suitable elastomeric materials include, but are not limited to, elastomeric block copolymers including the styrenic block copolymers such as styrene-ethylene/butylene-styrene (SEBS) block copolymers disclosed in U.S. Pat. No. 5,112,900 which is incorporated by reference herein in its entirety. Other suitable block copolymer elastomers include, but are not limited to, styrene-isoprene-styrene (SIS), styrene-butadiene-styrene (SBS), styrene-isobutylene-styrene (SIBS) and so forth. Block copolymer elastomers are also described in commonly assigned U.S. Pat. Nos. 6,406,457, 6,171,278, 6,146,356, 5,951,941, 5,830,182 and 5,556,383, each of which is incorporated by reference herein in its entirety.

Elastomeric polyesters and copolyesters may be employed herein. Examples of elastomeric copolyesters include, but are not limited to, poly(ester-block-ether) elastomers, poly(ester-block-ester) elastomers and so forth. Poly(ester-block-ether) elastomers are available under the trade name of HYTREL® from DuPont de Nemours & Co. and consist of hard segments of polybutylene terephthalate and soft segments based on long chain polyether glycols. These polymers are also available from DSM Engineering Plastics under the trade name of ARNITEL®.

Non-elastomeric polyesters and copolymers thereof may be employed, such as the polyalkylene naphthalates, including polyethylene terephthalate and polybutylene terephthalate, for example. Polyamides including nylon, and copolymers thereof, such as poly (ether-block-amides) available under the trade name of PEBAX® from Atofina Chemicals in Philadelphia, Pa., are suitable for use herein. Suitable balloon materials are described in commonly assigned U.S. Pat. Nos. 5,549,552, 5,447,497, 5,348,538, 5,550,180, 5,403,340 and 6,328,925, each of which is incorporated by reference herein in its entirety. The above lists are intended for illustrative purposes only, and shall not be construed as a limitation on the scope of the present invention.

Still referring to FIG. 1, it will be seen that the system 10 of the present invention includes a plurality of substantially straight blades 26, of which the blades 26a and 26b are only exemplary. As envisioned for the present invention, the system 10 may include only one such blade 26, or it may include more than one blade 26 (e.g. two, three or more). With this in mind, and using the blade 26a as a specific example for purposes of disclosure, it will be seen that the proximal end 28 of the blade 26a is positioned adjacent, or near, the proximal end 30 of the balloon 18. Further, it is to be appreciated that the blade 26a is oriented on the balloon 18 so that it is coplanar with the longitudinal axis 32 of the balloon 18 (see FIG. 2A). Also, it is to be appreciated by cross-referencing FIG. 1 with FIG. 2B, that the blade 26a is attached to a proximal section 34 of the balloon 18. For purposes of the present invention, the blades 26 can be attached to the balloon 18 by any means known in the art, such as by bonding.

The structure for balloon 18 will be best understood by referencing both FIG. 1 and FIG. 2B. As shown, the balloon 18, when inflated, generally defines three sections. These are: a proximal section 34; an intermediate section 36; and a distal section 38. More specifically, when the balloon 18 is inflated, the proximal section 34 is generally conical-shaped and has a taper with an increasing radius in the distal direction. On the other hand, the intermediate section 36 is substantially cylindrical-shaped and has a generally constant radius. Again, there is a conical-shape for the distal section 38. This time, however, the taper for the distal section 38 has a decreasing radius in the distal direction. Preferably, the blade 26a is longer than the proximal section 34 and is attached to only the proximal section 34 of the balloon 18. Consequently, the distal end 40 of the blade 26a is not engaged with the balloon 18. As perhaps best seen in FIG. 2B, this cooperation of structure allows the distal end 40 of the blade 26a, to extend radially outward from the axis 32 to a greater distance than the radius of the cylindrical-shaped, intermediate section 36. Stated differently, with the balloon 18 in its inflated configuration, the blades 26 are inclined at an angle (α) relative to the axis 32. Preferably, the angle (α) is in a range between 0° and 45°.

FIG. 1 also shows that the system 10 of the present invention can include radiopaque markers 44a and 44b which will assist in positioning the balloon 18 in the vasculature of the patient. Identification of the balloon 18 at a location in the vasculature can be further facilitated by using a contrast medium to inflate the balloon 18. Other mechanisms, known in the art, can be incorporated and used for these purposes.

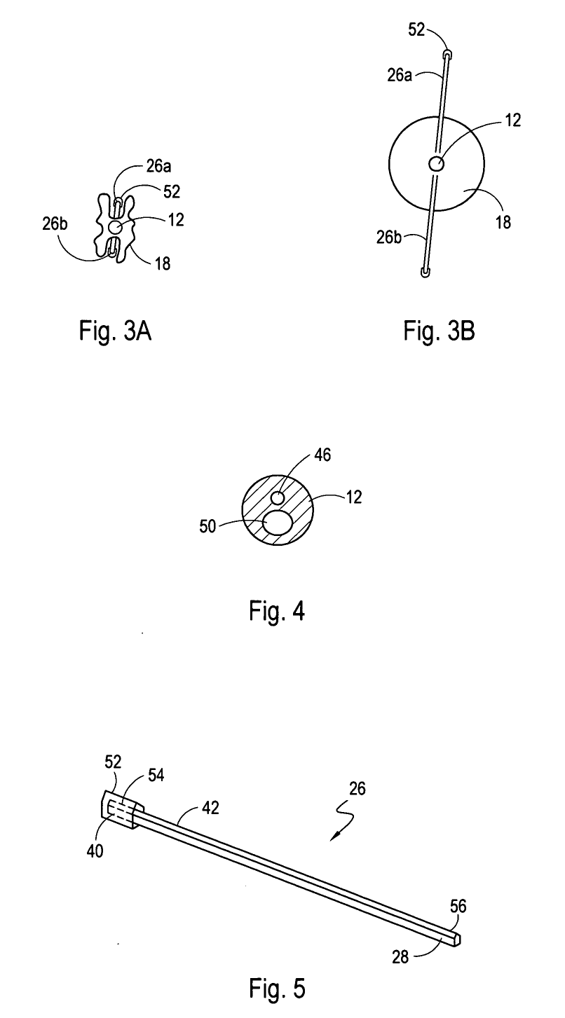

As envisioned for the present invention, the balloon 18 of the present invention can be reconfigured between a deflated configuration (FIG. 2A and FIG. 3A) and an inflated configuration (FIG. 2B and FIG. 3B). The actual inflation and deflation of the balloon 18 is accomplished by manipulating the inflation device 24 (See FIG. 1). Any type of balloon inflation device known in the art can be connected at the y-site 20 in fluid communication with an inflation lumen 46 (see FIG. 4). FIG. 4 also shows that the catheter 12 is formed with a guidewire lumen 50 for receiving the guidewire 22 therethrough.

As best seen in FIG. 5, each blade 26 includes a blunt tip member 52 at the distal end 40 of the blade 26 to prevent tissue incision by the distal end 40 of each blade 26 during a movement of the blade 26. For the embodiments shown in FIGS. 1-5, the blunt tip member 52 consists of a protective sheath that is positioned to overlay a distal portion of the cutting edge 42. Typically, the blunt tip member 52 extends between 5 and 50 percent of the length of the cutting edge 42. More specifically, for this embodiment, the cutting edge 42 extends from a cutting edge distal end 54 to a cutting edge proximal end 56 and the protective sheath overlays the cutting edge distal end 54. Also for this embodiment, the blunt tip member 52 can be a sheath that encapsulates a distal portion of the blade 26 (as shown) or a coating that is applied directly to the cutting edge 42 (not shown). In a particular embodiment, the protective sheath can be made of a polymeric material (e.g. plastic) and bonded to the portion of the blade 26 having the cutting edge 42.

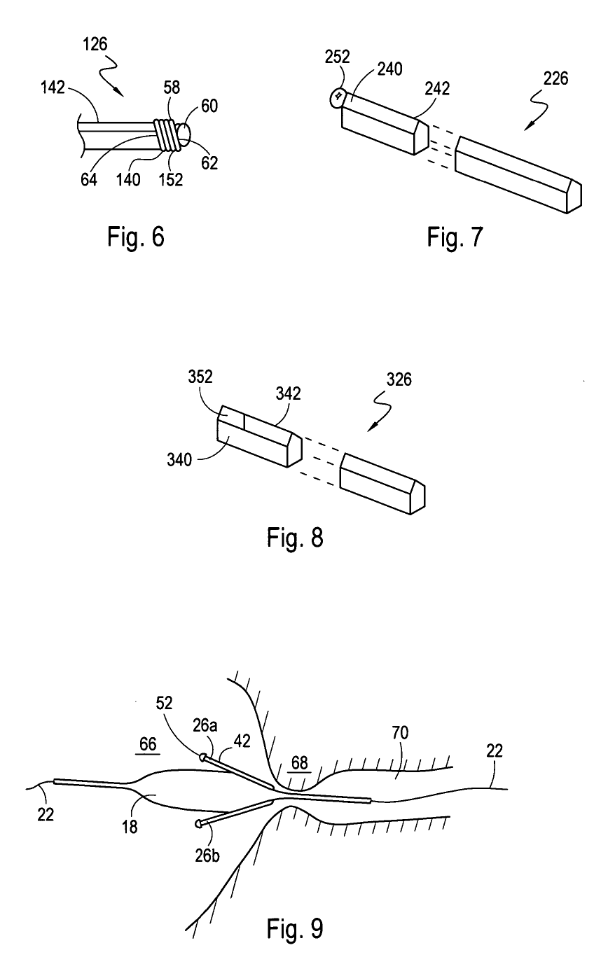

FIG. 6 shows another embodiment of a blade (designated 126) having a cutting edge 142 and blunt tip member 152 for use in the system 10 shown in FIG. 1. As shown in FIG. 6, the blunt tip member 152 is positioned at the distal end 140 of blade 126 and includes a fine coil wire 58 and ball shaped element 60. More particularly, FIG. 6 shows that the blunt tip member 152 can include a fine coil, helically wound wire 58 having a distal end 62 and a proximal end 64. The ball shaped element 60 is attached to the distal end 62 of the coil wire 58 and is typically formed having a spherical, spheroidal or hemispherical shape.

Referring now to FIG. 7, another embodiment of a blade (designated 226) is shown having a cutting edge 242 and blunt tip member 252 for use in the system 10 shown in FIG. 1. As shown in FIG. 7, the blunt tip member 252 is positioned at the distal end 240 of blade 226 and includes a spherical ball that is attached to the remaining portion of the blade 226. FIG. 8 shows still another embodiment of a blade (designated 326) having a cutting edge 342 and blunt tip member 352 for use in the system 10 shown in FIG. 1. As shown in FIG. 8, the blunt tip member 352 is positioned at the distal end 340 of blade 326 and is formed as a rounded surface to prevent tissue incision by the distal tip of the blade 326.

Referring now to FIG. 9, in the operation of the system 10 of the present invention, the guidewire 22 is pre-positioned in the vasculature of the patient. Although the treatment of an exemplary aortic valve stenosis is hereinafter described, it is to be appreciated that the system 10 can be used to incise tissue (including stenosed tissue) in other areas of the body. The catheter 12, with the balloon 18 in its deflated configuration (FIG. 2A and FIG. 3A) is then advanced over the guidewire 22. As intended for the operation of the present invention, the balloon 18 is advanced over the guidewire 22 until the balloon 18 has been positioned in the left ventricle 66 of the patient's heart. At this point, the inflation/deflation device 24 is manipulated to inflate the balloon 18 into its inflated configuration (FIG. 1, FIG. 2B and FIG. 3B). With the cutting blades 26 radially deployed, the balloon 18 is then withdrawn in a proximal direction through the aortic valve 68 and into the aorta 70. During withdrawal of the balloon 18, the cutting edges 42 of respective blades 26 incise the aortic valve 68 to relieve any stenosis that has developed in the aortic valve 68. After withdrawal, the balloon 18 is deflated, and the system 10 is removed from the vasculature of the patient.

While the particular Aortic Stenosis Cutting Balloon Blade and corresponding methods of use as herein shown and disclosed in detail are fully capable of obtaining the objects and providing the advantages herein before stated, it is to be understood that they are merely illustrative of the presently preferred embodiments of the invention and that no limitations are intended to the details of construction, design or use herein shown other than as described in the appended claims.

What is claimed is:

1. A cutting device comprising:
a catheter;
an elongated balloon defining an axis, the balloon being mounted on the catheter for reconfiguration between an inflated configuration and a deflated configuration;
an elongated blade formed with a cutting edge and having a distal end and a proximal end, with the proximal end thereof attached to the balloon to orient the blade in a plane with the axis of the balloon and to project the cutting edge of the blade in a radial direction from the axis of the balloon; and
a blunt tip member at the distal end of the blade to prevent tissue incision by the distal end of the blade during a movement of the blade.

2. A device as recited in claim 1 further comprising a guidewire for advancing the catheter through the vasculature of a patient to position the balloon for cutting tissue at a predetermined site in the vasculature.

3. A device as recited in claim 2 wherein the predetermined site in the vasculature is the valve between the left ventricle of the heart and the aorta.

4. A device as recited in claim 1 wherein the balloon has a proximal end and a distal end and, in its inflated configuration, the balloon further comprises:
a substantially conical-shaped, proximal section having a taper with increasing radius in the distal direction;
a substantially conical-shaped, distal section having a taper with decreasing radius in the distal direction;
a substantially cylindrical-shaped, intermediate section located between the proximal section and the distal section; and
wherein the blade is attached to the proximal section of the balloon with the proximal end of the blade adjacent the proximal end of the balloon.

5. A device as recited in claim 1 wherein the blunt tip member comprises:
a coil wire having a distal end and a proximal end with the proximal end thereof connected to the distal end of the blade; and
a ball shaped element connected to the distal end of the coil wire.

6. A device as recited in claim 1 wherein the cutting edge extends from a cutting edge distal end to a cutting edge proximal end and the blunt tip member comprises a protective sheath overlaying the distal end of the cutting edge.

7. A device as recited in claim 6 wherein the protective sheath is made of plastic.

8. A device as recited in claim 1 wherein the blunt tip member has a surface and at least a portion of the surface is rounded.

9. A device for incising tissue, the device comprising:
a substantially straight, elongated blade having a distal end and a proximal end, with a cutting edge located therebetween;
a blunt tip member at the distal end of the blade;
a means for advancing the elongated blade in a distal direction along a predetermined path into the vasculature of a patient;
a means for inclining the blade relative to the path with an increasing distance between the blade and the path in the distal direction; and
a means for withdrawing the blade in a proximal direction along the path to incise the tissue with the cutting edge of the blade.

10. A device as recited in claim 9 wherein the advancing means is a catheter and the inclining means is a balloon.

11. A device as recited in claim 10 wherein the balloon has a proximal end and a distal end and, when inflated, the balloon further comprises:
a substantially conical-shaped, proximal section having a taper with increasing radius in the distal direction;
a substantially conical-shaped, distal section having a taper with decreasing radius in the distal direction; and
a substantially cylindrical-shaped, intermediate section located between the proximal section and the distal section.

12. A device as recited in claim 11 wherein the blade is attached to the proximal section of the balloon with the proximal end of the blade adjacent the proximal end of the balloon.

13. A device as recited in claim 9 wherein the blunt tip member comprises:
a coil wire having a distal end and a proximal end with the proximal end thereof connected to the distal end of the blade; and
a ball shaped element connected to the distal end of the coil wire.

14. A device as recited in claim 9 wherein the cutting edge extends from a cutting edge distal end to a cutting edge proximal end and the blunt tip member comprises a protective sheath overlaying the distal end of the cutting edge.

15. A device as recited in claim 14 wherein the protective sheath is made of plastic.

16. A device as recited in claim 9 wherein the blunt tip member has a surface and at least a portion of the surface is rounded.

17. A method for incising target tissue with a blade, the method comprising the steps of:
providing a substantially straight, elongated blade having a distal end and a proximal end, a cutting edge located therebetween, and with the blade having a blunt tip member at the distal end thereof to prevent tissue incision by the distal end during a movement of the blade;
advancing the elongated blade in a distal direction along a predetermined path into the vasculature of a patient, to position the blade distal to the target tissue;
inclining the blade relative to the path with an increasing distance between the blade and the path in the distal direction; and
withdrawing the blade in a proximal direction along the path to incise the target tissue with the cutting edge of the blade.

18. A method as recited in claim 17 wherein the blunt tip member comprises a coil wire and a ball shaped element, the coil wire having a distal end and a proximal end with the ball shaped element located at the distal end of the coil wire.

19. A method as recited in claim 17 wherein the cutting edge extends from a cutting edge distal end to a cutting edge proximal end and the blunt tip member comprises a protective sheath overlaying the distal end of the cutting edge.

20. A method as recited in claim 17 wherein the blunt tip member has a surface and at least a portion of the surface is rounded.
